(19) United States
(12) Patent Application Publication
(10) Pub. No.: US 2020/0055914 A1
(43) Pub. Date: Feb. 20, 2020

(54) Stabilization of Insulin Self-Assembly by B26 Aromatic Substitutions

(71) Applicant: Case Western Reserve University, Cleveland, OH (US)

(72) Inventors: Michael A. WEISS, Indianapolis, IN (US); Nelson PHILLIPS, Seven Hills, OH (US); Nischay REGE, Cleveland, OH (US)

(21) Appl. No.: 16/425,906

(22) Filed: May 29, 2019

Related U.S. Application Data
(60) Provisional application No. 62/677,634, filed on May 29, 2018.

(51) Int. Cl. C07K 14/62

(52) U.S. Cl. CPC C07K 14/62; A61K 38/00

(57) ABSTRACT

An insulin analogue comprises an insulin B-chain polypeptide containing a Trp substitution at position B26 relative to the sequence of wild-type insulin. The insulin analogue may additionally comprise an Orn^{B29} substitution, a C-terminal extension of one or two basic amino acids such as Arg-Arg, a Gln^{B13} substitution, a Gly^{A21} substitution, a His^{A8} or Arg^{A8} substitution, or a combination thereof. The insulin analogue may be formulated in the presence of zinc ions at a molar ratio of 2.2-10 zinc ions per six insulin analogue monomers. The molecular design is believed to stabilize the dimer interface of insulin (and its stable formulation as a zinc insulin hexamer) by means of aromatic amino-acid substitutions at position B26 of the B chain. The insulin analogs of the present invention may have two chains (A and B) as in mammalian insulins or may be engineered with a C domain (4-12 amino acids in length) to provide a single-chain. The TrpB26-stabilized zinc insulin hexamers complement and extend other molecular strategies to achieve protracted action on subcutaneous injection.

### CROSS REFERENCE TO RELATED APPLICATIONS

This application claims benefit of co-pending U.S. Provisional Application No. 62/677,634 filed on May 29, 2018.

### STATEMENT REGARDING FEDERALLY SPONSORED RESEARCH OR DEVELOPMENT

This invention was made with government support under grant number DK040949 awarded by the National Institutes of Health. The government has certain rights in the invention.

### BACKGROUND OF THE INVENTION

This invention relates to polypeptide hormone analogues that exhibits enhanced pharmaceutical properties, such as increased thermodynamic stability, augmented resistance to thermal fibrillation above room temperature, decreased mitogenicity, and/or altered pharmacokinetic and pharmacodynamic properties, i.e., conferring more prolonged duration of action or more rapid duration of action relative to soluble formulations of the corresponding wild-type human hormone. More particularly, this invention relates to insulin analogues containing a substitution at position B26 of the insulin B chain whereby the native Tyrosine is replaced by an alternative aromatic amino acid (natural or unnatural) that confers enhanced stability to the dimer interface and/or that prolongs the lifetime of an insulin hexamer in a pharmaceutical formulation. Such substitutions will be useful in enhancing the pharmacologic properties of long-acting (or basal) insulin analogue formulations.

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often confer multiple biological activities. A benefit of non-standard proteins would be to achieve more prolonged action, leading to a flatter pharmacokinetic (PK) or pharmacodynamic (PD) profile following once-a-day administration or even enabling development of once-a-week administration. An example of a therapeutic protein is provided by insulin. Wild-type human insulin and insulin molecules encoded in the genomes of other mammals bind to insulin receptors is multiple organs and diverse types of cells, irrespective of the receptor isoform generated by alternative modes of RNA splicing or by alternative patterns of post-translational glycosylation.

An example of a further medical benefit would be optimization of the thermodynamic or kinetic stability of a protein assembly toward dissociation. Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinopathy, blindness, and renal failure. Critical to the safe and convenient achievement of tight glycemic control by patients with Type 1 diabetes mellitus and by a subset of patients with Type 2 diabetes mellitus has been the development of novel insulin analogues that differ in sequence from naturally occurring mammalian insulins due to the presence of amino-acid substitutions or modified amino-acid side chains. Such substitutions and modifications have been introduced in the art to make rapid-acting insulin formulations even more rapid and to make long-acting insulin formulations even longer acting. These two classes of analogues are respectively known as prandial insulin analogue formulations and basal insulin analogue formulations.

Insulin is a small globular protein that plays a central role in metabolism in vertebrates. Insulin contains two chains, an A chain, containing 21 residues, and a B chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain (FIG. 1A). A variety of evidence indicates that it consists of an insulin-like core and disordered connecting peptide (FIG. 1B). Formation of three specific disulfide bridges (A6-A11, A7-B7, and A20-B19; FIGS. 1A and 1B) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). The sequence of insulin is shown in schematic form in FIG. 1C. Individual residues are indicated by the identity of the amino acid (typically using a standard three-letter code), the chain and sequence position (typically as a superscript). Pertinent to the present invention is the presence of a conserved triplet of aromatic amino acids in the B chain (Phe^{B24}, Phe^{B25} and Tyr^{B26}). These aromatic side chains pack at or adjoin the dimer interface of insulin, which occurs three times in the structure of an insulin hexamer (FIG. 2). On binding of an insulin monomer to the insulin receptor, they also contact the hormone-binding surface of the receptor ectodomain.

The present invention was motivated by the medical and societal needs to engineer basal once-a-day single-chain insulin analogues that exhibit delayed pharmacokinetic (PK) properties in the subcutaneous depot. Three existing methods are known in the art. (i) The first employs “iso-electric precipitation” to convert a soluble pharmaceutical formulation at pH 3.0-4.5 to an insoluble subcutaneous precipitate or microcrystalline suspension on injection to the neutral-pH environment of the subcutaneous space. An example is provided by insulin glargine (the active component of products Lantus® and Toujeo®; Sanofi), which contains a di-Arginine extension of the B-chain at positions B31 and B32 (Arg^{B31} and Arg^{B32}). (ii) The second method employs acylation of the epsilon-amino group of a Lysine side chain at position B29 of human insulin, such as by myrstic acid or by a 16-carbon fatty di-carboxylic acid attached via a glutamic acid spacer. These modifications are respectively found in insulin detemir and insulin degludec (the active components of products Levemir® and Tresiba®; Novo-Nordisk). Such modifications can stabilize multi-hexamer assemblies in the SQ depot and also mediate binding in the bloodstream to serum albumin. (iii) The third method employs polyethylene glycol polymers as may be attached to the epsilon-amino group of Lysine at either position B29 of human insulin or position B28 of an analogue known in the art as insulin lispro (PEGylated insulin lispro; Eli Lilly and Co.; withdrawn from human clinical trials due to hepatotoxicity). None of these prior strategies exploits the structure of the zinc insulin hexamer itself to delay its dissociation into zinc-free dimers and monomers. Such dissociated dimers and monomers are the species primarily responsible for passage of the insulin molecule out of the subcutaneous space and into the bloodstream.

It would be desirable, therefore, to invent a novel class of insulin analogues whose self-assembly as a zinc insulin hexamer is stabilized on a thermodynamic or kinetic basis, such that dissociation of the hexamer in the subcutaneous is delayed. More generally, there is a need for molecular design strategy to delay of the absorption of human insulin by a new mechanism or to further prolong the absorption of basal insulin analogues as known in the art.

### SUMMARY OF THE INVENTION

It is, therefore, an aspect of the present invention to provide a substitution or class of aromatic substitutions at position B26 in wild-type human insulin or in insulin analogues such that the dimer interface is stabilized and/or that the lifetime of the zinc insulin analogue hexamer is prolonged. This position in the B chain of mammalian insulins (and indeed in almost all vertebrate insulins) is conserved as Tyrosine. In the three-dimensional structure of the zinc-free insulin dimer or zinc insulin hexamer, this Tyrosine (Tyr^{B26}) and its dimer-related mate participate in a cluster of aromatic rings at the dimer interface, including also Tyr^{B16}, Phe^{B24} and their respective dimer-related mates; the aromatic side chain of Phe^{B25} is more distant (FIG. 2). Although not wishing to be restricted by theory, successive edge-to-face interactions among these six aromatic rings (B16, B24, B26 and their dimer-related mates; FIG. 3) and their burial within a non-polar environment appear to stabilize the dimer interface (FIG. 2). Aromatic substitutions larger than the phenolic moiety of Tyrosine may thus enhance one or both of these contributions to dimer stability while preserving at least a portion of the hormone's biological activity. In one example the modified B chain contains substitution Tyr^{B26}→Trp, whose indole ring is larger than the phenolic moiety of Tyr and which may confer more favorable aromatic-aromatic interactions at the dimer interface. A diversity of unnatural amino-acid side chains may function as well as Tryptophan at B26 to stabilize the insulin dimer and R₆ zinc insulin hexamer.

In general, the invention provides an insulin analogue that comprises an insulin B-chain polypeptide containing a substitution at position B26 relative to the sequence of wild-type insulin selected from Trp or a non-naturally occurring aromatic amino acid residue. The insulin analogue may additionally comprise an Orn^{B29} substitution, a C-terminal extension of one or two basic amino acids such as Arg-Arg, a Gln^{B13} substitution, a Gly^{A21} substitution, a His^{A8} or Arg^{A8} substitution, or a combination thereof. In addition or in the alternative, the insulin analogue may comprise paired His A4-HisA8 substitutions, optionally with a Gly or Ala substitution at position A21. In a further example, the insulin analogue may comprise a Gln^{B13} substitution, optionally with His or Arg at position A8 and optionally with Gly or Ala at position A21. In still another example, the insulin analogue may comprise a Lys^{B29} modified by an acyl group or by a fatty dicarboxylic acid (via a glutamic acid spacer) and which contains a substitution of Tyr^{B26} by Trp or by a non-naturally occurring aromatic amino-acid residue.

The insulin analogue may be formulated in the presence of zinc ions at a molar ratio of 2.2-10 zinc ions per six insulin analogue monomers, and at successive strengths U-100 to U-1000 in soluble solutions at at least a pH value in the range 3.0-4.5. In other examples, the insulin analogue may be formulated in the presence of zinc ions at a molar ratio of 2.0-3.0 zinc ions per six insulin analogue monomers, and at successive strengths U-100 to U-1000 in soluble solutions at at least a pH value in the range 6.5-8.0. The insulin analogues of the present invention may have two chains (A and B) as in mammalian insulins or may be engineered with a connecting C domain (4-12 amino acids in length) between the A-chain and the B-chain to provide a single-chain insulin analogue.

A method of lowering the blood sugar level of a patient, such as a patient with diabetes mellitus, comprises administering a physiologically effective amount of the insulin analogue or a physiologically acceptable salt thereof to a patient.

### BRIEF DESCRIPTION OF THE SEVERAL VIEWS OF THE DRAWINGS

FIG. 1A is a schematic representation of the sequence of human proinsulin (SEQ ID NO: 1) including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin indicating the position of various residues in the A-chain (SEQ ID NO: 2) and B-chain (SEQ ID NO:3).

FIG. 2A provides a sequence of insulin with disulfide bridges in black. Tyr^{B26} is highlighted in black, the present Tyr^{B26}_Trp substitution in gray; and modifications in pI-shifted clinical analogue glargine as C-terminal extension Arg^{B31} and Arg^{B32}. Our semisynthetic pI-shifted analogue contained Orn (below main sequence) instead of Lys or Arg.

FIG. 2B provides a model of the structure of an insulin monomer. The A chain is shown in black and B chain in gray. Tyr^{B26} is dark gray whereas Phe^{B24} and Tyr^{B16} are medium gray (PDB: 4INS).

FIG. 2C shows a depiction of the Zn-coordinated insulin hexamer (T₆ state), a trimer of dimers; Tyr^{B26}, Phe^{B24} and Tyr^{B16} are color-coded as in FIG. 2B.

FIG. 2D provides a stereo view showing Tyr^{B26} (sticks) in a cavity within insulin dimer (extracted from T₃R^{f}₃ hexamer 1TRZ).

FIG. 2E provides a stick model corresponding to the FIG. 2D.

FIG. 3A provides molecular simulations of aromatic interactions in the insulin dimer. involving Phe^{B24}, Tyr^{B16}, Phe^{D24} (sticks) and either Tyr^{D26} (left) or Trp^{D26} (right). Residues were extracted from T₆ structure 4INS.

FIG. 3B provides contour maps depicting empirical interaction energies between B26 (Tyr on left and Trp on right) at varying χ₁ and χ₂ angles and the other three residues shown in A. The orientation of Tyr^{B26} in WT crystal structure is indicated by an “x”; orientation of Trp^{B26} in naive model is indicated by an asterisk.

FIG. 4A provides a ball-and-stick model of a tetrahedral Zn2+-coordination site in R6 insulin hexamer.

FIG. 4B provides a graph of the absorbance spectra of d-d bands in a corresponding Co2+complex in wild type insulin (SEQ ID NOs: 2 and 3), Orn^{B29} insulin analogue (SEQ ID Nos: 2 and 12), Lys^{B28}, Pro^{B29} (Lispro) insulin (SEQ ID NOs: 2 and 14), and Trp^{B26}, Orn^{B29} insulin analogue (SEQ ID NOs: 2 and 13).

FIG. 4C is a graph of absorbance at 574 nm over time after addition of excess EDTA showing hexamer dissociation; the symbols are as in FIG. 4B. The lifetime of the TrpB26, OrnB29-insulin hexamer was markedly prolonged (asterisk).

FIG. 4D is a graph of absorbance at 574 nm over time after addition of excess EDTA showing hexamer dissociation of Trp^{B26}, Orn^{B29} hexamer from 0-8000 sec in relation to that of parent OrnB29-insulin (black arrow). Half-lives are given in Table 1.

FIG. 5A presents size-exclusion chromatography (SEC) of Trp^{B26} hexamer (SEQ ID NOs: 2 and 4) as a SEC chromatogram of insulin analogues in the presence of zinc and phenol. The void volume (V₀, arrow) was defined by thyroglobulin (MW 669 kDa).

FIG. 5B is a graph of the log (molecular weight) vs elution ratio (Vₑ/V₀) of molecular weight standards. Linear relationship between log[MW] to elution ratio (Vₑ/V₀) is indicated by the line with coefficient of determination (R²) 0.996 and parameters log[molecular weight]=−1.71*(Vₑ/V₀)+6.7012. Elution times of molecular weight standards are indicated by squares (labeled by molecular weight). Identity of molecular-weight standards is as follows: 66 kDa, BSA; 45 kDa ovalbumin; 20 kDa, carbonic anhydrase, 17 kDa, myosin light chain; 12.4; cytochrome C, 6.5 IGF-I. Calculated MW are given in Table 1.

FIG. 6A is a graph of the receptor binding affinities (isoform B) of wild type insulin (SEQ ID NOs: 2 and 3), Orn^{B29} insulin analogue (SEQ ID NOs: 2 and 12), and Trp^{B26}, Orn^{B29} insulin analogue (SEQ ID NOs: 2 and 13).

FIG. 6B is a graph of blood glucose levels over time following intravenous (IV) injection in rats (N=15) of wild type insulin (SEQ ID NOs: 2 and 3), Orn^{B29} insulin analogue (SEQ ID NOs: 2 and 12), and Trp^{B26}, Orn^{B29} insulin analogue ((SEQ ID NOs: 2 and 13); symbols as in FIG. 6A).

FIG. 6C is a graph of blood glucose levels over time following subcutaneous (SQ) injection of Orn^{B29} insulin analogue (SEQ ID NOs: 2 and 12), and Trp^{B26}, Orn^{B29} insulin analogue (SEQ ID NOs: 2 and 13), in absence or presence of 0.3 mM ZnCl₂ (N=18).

FIG. 6D is a histogram summarizing the rate of fall of [blood-glucose] over first 30 min in FIG. 6C (black bars indicate S.D.).

FIG. 6E is a graph of the [blood glucose] level over time following SQ injection of pI-shifted analogs: Gly^{A21}, Orn^{B29}, Orn^{B31}, Orn³²-insulin (SEQ ID NOs: 17 and 15) and its Trp^{B26} derivative (SEQ ID NOs: 17 and 16; N=6).

FIG. 7A is a depiction of the electron density of Trp^{B26} insulin in a T-state protomer showing surrounding density in TR^{f} asymmetric unit (contour level 2.0 Å).

FIG. 7B is a stick model of the depiction of FIG. 7A.

FIG. 7C is a depiction of the surfaces of residues surrounding Trp^{B26} (sticks) as in FIGS. 7A and 7B.

FIG. 7D is a depiction of the electron density of Trp^{B26} insulin in an R-state protomer showing surrounding density in TR^{f} asymmetric unit (contour level 2.0 Å).

FIG. 7E is a stick model of the depiction of FIG. 7D.

FIG. 7F is a depiction of the surfaces of residues surrounding Trp^{B26} (sticks) as in FIGS. 7D and 7E.

FIG. 8A is a CD spectra of Trp^{B26}, Orn^{B29}-insulin (SEQ ID NOs: 2 and 13), Orn^{B29}-insulin (SEQ ID NOs: 2 and 12), and WT insulin (SEQ ID NOs: 2 and 3).

FIG. 8B is a depiction of the results of guanidine denaturation assays of insulin analogues monitored by ellipticity at 222 nm; symbol code as in FIG. 8A. Stabilities are given in Table 2.

FIG. 9A is depiction of the homonuclear 2D-NMR spectra of parent monomer insulin lispro (Lys^{B28}, Pro^{B29}-insulin; SEQ ID NOs: 2 and 14) showing the aromatic region of TOCSY spectrum with Tyr^{B26} cross peaks (magenta) shown relative to Tyr spin system in free octapeptide GFFYTKPT (dotted lines). TOCSY mixing time was 55 ms.

FIG. 9B is depiction of the homonuclear 2D-NMR spectra of parent monomer insulin lispro (Lys^{B28}, Pro^{B29}-insulin; SEQ ID NOs: 2 and 14) showing the region of NOESY spectrum showing contacts between aromatic protons (vertical axis, ω₂) and methyl groups (horizontal axis, ω₁). NOESY mixing time was 150 ms.

FIG. 9C is a depiction of the homonuclear 2D-NMR spectra of the Trp^{B26} analogue of insulin lispro showing the aromatic TOCSY spectrum highlighting Trp^{B26} cross peaks (red) relative to Trp spin system in free octapeptide GFFWTKPT (dashed lines). TOCSY mixing time was 55 ms.

FIG. 9D is depiction of the homonuclear 2D-NMR spectra of the Trp^{B26} analogue of insulin lispro showing the region of NOESY spectrum corresponding to FIG. 9B. B26-related NOEs are shown in red. Cross-peak assignments: (a) γ-CH₃ Val^{A3}, (b) γ-CH₃ Val^{B12}, (c) γ-CH₂, γ-CH₃ Ile^{A2}, (d) δ-CH₃ Leu^{B15}, (e) γ-CH₃ Val^{A3}, (f) γ-CH₃ Val^{B12}, (g) γ-CH₂, γ-CH₃ Ile^{A2}, (h) δ-CH₃ Ile^{A2}, and (i) δ-CH₃ Leu^{B15}. NOESY mixing time was 150 ms.

FIG. 10A is a model of WT insulin (in classical T-state) overlaid on structure of insulin bound to an IR fragment (PDB entry 4OGA), depicting the binding surface of Tyr^{B26} as bound to a receptor ectodomain fragment. The L1 domain and part of CR domain are shown; the tube indicates classical location within overlay of residues B20-B30 (arrow), thereby highlighting steric clash of B26-B30 with αCT. Insertion of the insulin B20-B27 segment between L1 and αCT was associated with a small rotation of the B20-B23 β-turn and changes in main-chain dihedral angles flanking B24.

FIG. 10B is a stick representation of B-chain residues B20-B27 packed between αCT and the L1 β2 strand. Residues B8-B19 are shown as a black ribbon, and the A chain is shown as a yellow ribbon. Key contact surfaces of αCT with B24-B26 are highlighted in magenta and of L1 with B24-B26 are highlighted.

FIG. 10C is a stereo view of environment of Tyr^{B26} within its binding site. Neighboring side chains in L1 and αCT are as labeled.

FIG. 11A is an isosurface representation of electron density and molecular electrostatic potential (MEP) map of Tyr (left) and Trp (right), comparing the ab initio electrostatics and CHARMM parameters of Tyr and Trp side chains. Electron density and MEP were calculated using B3LYP method and 6-31G(d) basis set using Gaussian utility Cubegen on Gaussian 09. The isosurface map was then generated using Jmol.

FIG. 11B provides ball-and-stick models of Tyr (left) and Trp (right) side chains. Point charges of each atom as implemented in CHARMM22 are indicated.

FIG. 12A is an SEC profile of monomeric lispro (Zn²⁺- and phenol-free). The molecular weight (MW; or molecular mass) of the species was 3.1 kDa (calculated as in FIG. 5 above).

FIG. 12B is an SEC profile of WT insulin formulated with 0.3 mM ZnCl₂ and phenol was run in a mobile-phase containing 50 mM cyclohexanol and 0.3 mM ZnCl₂. The sample eluted as a hexamer (Calculated molecular mass 48 kDa) with dissociation intermediates constituting the “tail” of the peak.

FIG. 12C is a calibration plot of the SEC column with mobile phase used in FIG. 12B. Linear fit (line) of log(MW) to Vₑ/V₀ of MW standards (squares). The equation of the line is log(MW)=−1.79 (Vₑ/V₀)+6.83 (R²=0.986). The following standards were used for calibration: thyroglobulin (669 kDa, V₀), ovalbumin (45 kDa), carbonic anhydrase (29 kDa), elastase (26 kDa), ribonuclease A (13.7 kDa), cytochrome C (12.3 kDa) and synthetic peptides (3.6 and 1.2 kDa).

FIG. 13A is a graph of blood glucose concentration over time after SQ injection of WT insulin (SEQ ID NOs: 2 and 3) formulated in absence of Zn²⁺ (N=8) or in presence of 0.3 mM ZnCl₂ (N=9).

FIG. 13B is a graph showing the normalized blood glucose levels shown in FIG. 13A.

FIG. 14A is a graph of the normalized blood glucose concentration over time after IV injection of parent pI-shifted Gly^{A21}, Orn^{B29}, Orn^{B31}, Orn^{B32} insulin analogue (SEQ ID NOs: 17 and 15; triangles; N=6) and its Trp^{B26} derivative (SEQ ID NOs: 17 and 16; squares; N=6).

FIG. 14B is a bar graph showing the area over curve (AOC) from FIG. 14A. Bars indicate S.E.M. The Trp^{B26} derivative displayed 82±6% potency relative to the parent analog but is a complete agonist on injection of higher doses.

FIG. 15A is a graph of blood glucose concentration over time after SQ injection of zinc-free parent pI-shifted insulin analog (SEQ ID NOs: 17 and 15; squares; N=6) and Trp^{B26} derivative (SEQ ID NOs: 17 and 16; triangles; N=6).

FIG. 15B is a graph showing the normalized blood glucose levels shown in FIG. 15A.

FIG. 15C is a graph of blood glucose concentration over time after SQ injection parent pI-shifted insulin analog (SEQ ID NOs: 17 and 15; squares; N=6) and Trp^{B26} derivative (SEQ ID NOs: 17 and 15; triangles; N=6) formulated in the presence of 0.3 mM ZnCl₂.

FIG. 15D is a graph showing the normalized blood glucose levels shown in FIG. 15C.

FIG. 16A is a ribbon model of an R^{f}-state protomer of Trp^{B26}, Orn^{B29}-insulin (SEQ ID NOs: 2 and 13).

FIG. 16B is a stick model of an R^{f}-state protomer of Trp^{B26}, Orn^{B29}-insulin (SEQ ID NOs: 2 and 13; sticks) in relation to extensive set of crystal structures of insulin and insulin analogs (PDB entries: 1BEN, 1G7A, 1RWE, 1EV3, 1EV6, 1MPJ, 1TRZ, 1TYL, 1MPJ, 1ZEG, 1ZNJ and 1ZNI; gray sticks). Structures are aligned with respect to the main-chain atoms of residues A1-A21 and B3-B28.

FIG. 16C is a ribbon model of a T-state protomer of Trp^{B26}, Orn^{B29}-insulin (SEQ ID NOs: 2 and 13).

FIG. 16D is a stick model of a T-state protomer of Trp^{B26}, Orn^{B29}-insulin (SEQ ID NOs: 2 and 13; sticks) in relation to extensive set of crystal structures of insulin and insulin analogs. PDB entries used for alignment are as follows: 1APH, 1DPH, 1BEN, 1MPJ, 1TRZ, 1TYL, 1TYM, 1RWE, 1G7A, 1ZNI, 2INS and 4INS.

FIG. 17 provides ball-and-stick models and ab initio calculations of energy of interaction between pairs of isolated aromatic molecules: phenol-phenol (top), phenol-benzene (middle), and phenol-indole (bottom). The phenol-indole pair was determined to form the most stable ETF interaction as a result of Van der Waals forces. Interaction energies were calculated using MP2 method and aug-cc-pVDZ basis set using Gaussian 09.

FIG. 18A is a structural representation of a T₆ insulin hexamer. The eight N-terminal residues of the B chain (light gray) are in an extended conformation (box).

FIG. 18B is a structural representation of a R₆ insulin hexamer stabilized by bound phenolic ligands (blue). Residues B1-B8 form an α-helix (box).

FIG. 18C is a structural representation providing the location of GlyB⁸ which may serve as the pivot point of the transition between the R- and T-states.

FIG. 18D is a structural representation providing the location of GlyB⁸. GlyB⁸ is thought to adopt a right-handed conformation (i.e., with positive φ angle) in the T-state (light gray) and a left-handed conformation (negative φ angle) in the R-state (dark gray).

FIG. 19A is a ribbon model displaying the orientation of Phe^{B25} in the insulin dimer (PDB 4INS) in a first view. Phe^{B25} (dark gray sticks) is peripheral to the aromatic network formed by Tyr^{B16}, Phe^{B24}, Tyr^{B26} and their symmetry-related mates (light gray sticks).

FIG. 19B is a ribbon model displaying the orientation of Phe^{B25} in the insulin dimer (PDB 4INS) in a second view. Phe^{B25} (dark gray sticks) is peripheral to the aromatic network formed by Tyr^{B16}, Phe^{B24}, Tyr^{B26} and their symmetry-related mates (light gray sticks).

FIG. 20A is a stereo view of the B26 crevice within TR^{f} dimers of Trp^{B26}, Orn^{B29}-insulin and WT (1TRZ). Tyr^{B26} (sticks) of 1TRZ T-state protomer within an electrostatic potential surface (generated using APBS plug-in to Pymol®) formed by the surrounding residues.

FIG. 20B is a stereo view of the B26 crevice in a first possible orientation of Trp^{B26} that retain χ₁ and χ₂ angles of the WT structure shown in FIG. 20A.

FIG. 20C is a stereo view of the B26 crevice in a second possible orientation of Trp^{B26} that retain χ₁ and ω₂ angles of the WT structure shown in FIG. 20A. Due to the asymmetric structure of the Trp side chain, two possible ω₂ angles of correspond in principle to the native Tyr. However, Trp^{B26} encounters a steric clash with residues in the core of insulin in the orientation shown.

FIG. 20D is a stereo view of the B26 crevice showing Trp^{B26} of the R^{f}-state protomer from the Trp^{B26}, Orn^{B29}-insulin crystal structure depicted within an electrostatic potential surface formed by surrounding residues.

FIG. 20E is a stereo view of Trp^{B26} from FIG. 20D within the B26 crevice of FIG. 20A (WT). The Trp^{B26} side chain does not encounter a steric clash.

FIG. 20F is a stereo view of the alignment of the naïve model of Trp^{B26} from FIG. 20C to the Trp^{B26}, Orn^{B29}-insulin structure. Residues are depicted within the WT crevice (FIGS. 20A, 20B, 20C, and 20E). The steric clash predicted in the naïve model is mitigated in the Trp^{B26}, Orn^{B29}-insulin structure by (a) a local shift (0.2 Å) in the backbone of the C-terminal B-chain and (b) a slight difference in the χ₁ torsion angle of Trp^{B26}.

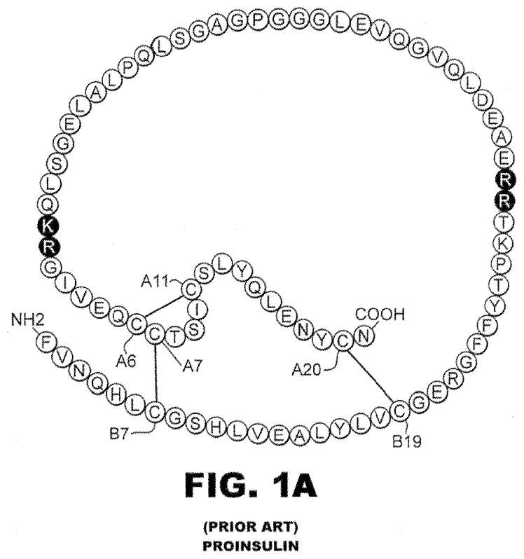

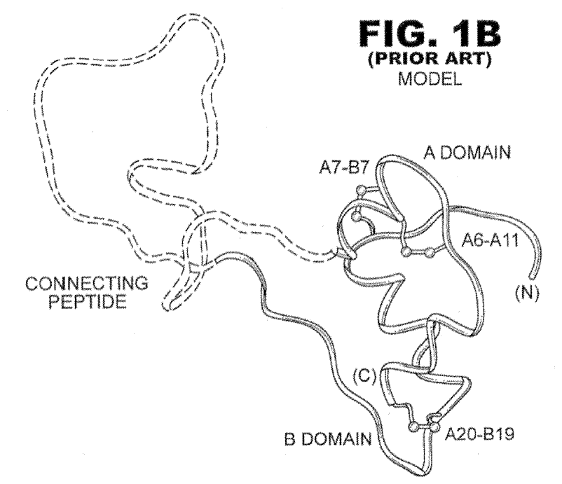

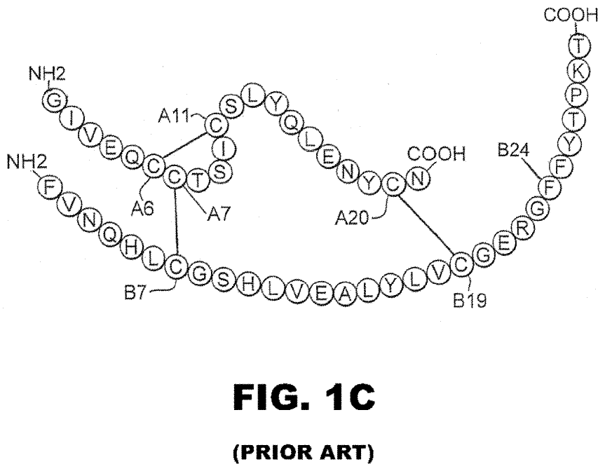

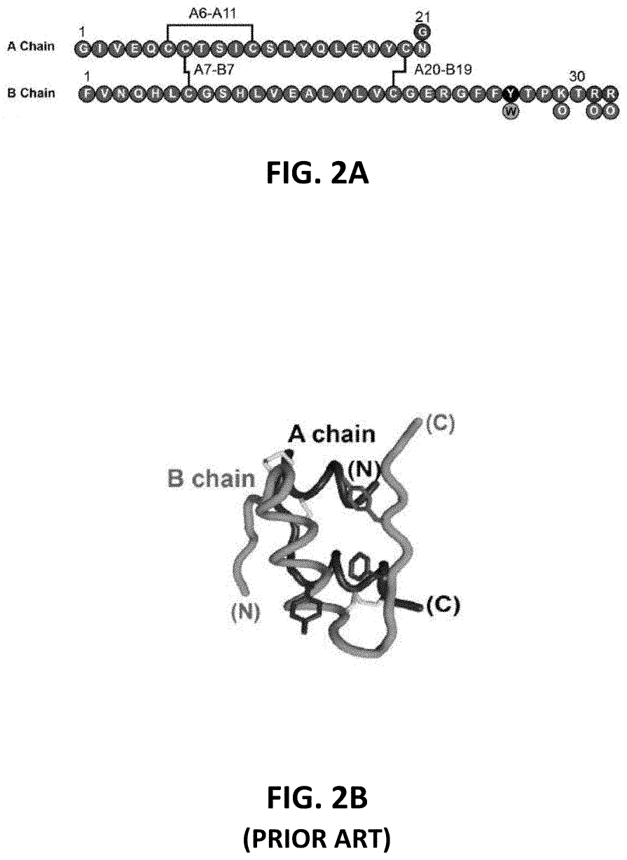

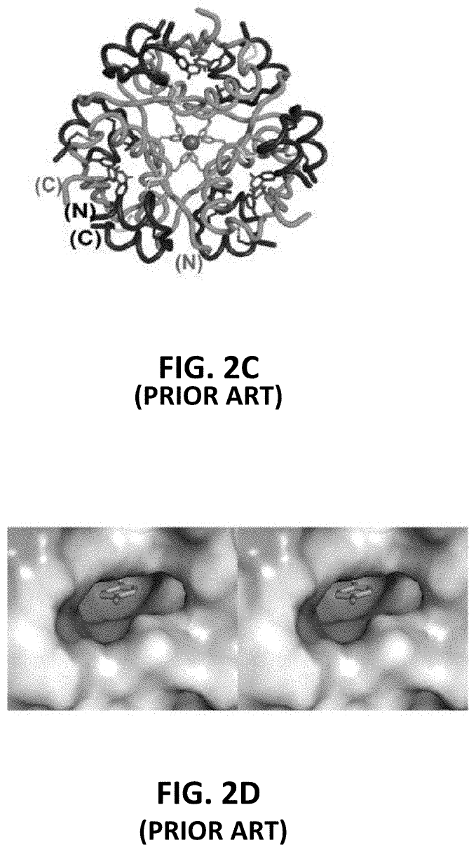

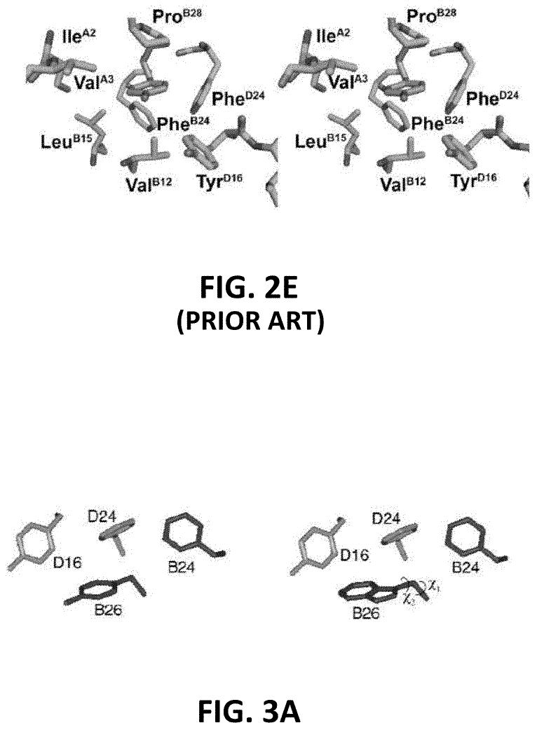

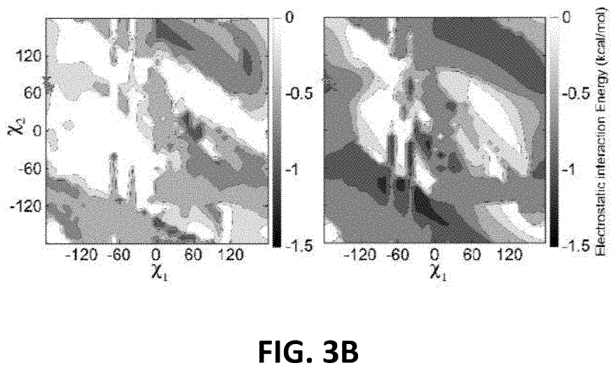

### DETAILED DESCRIPTION OF THE INVENTION

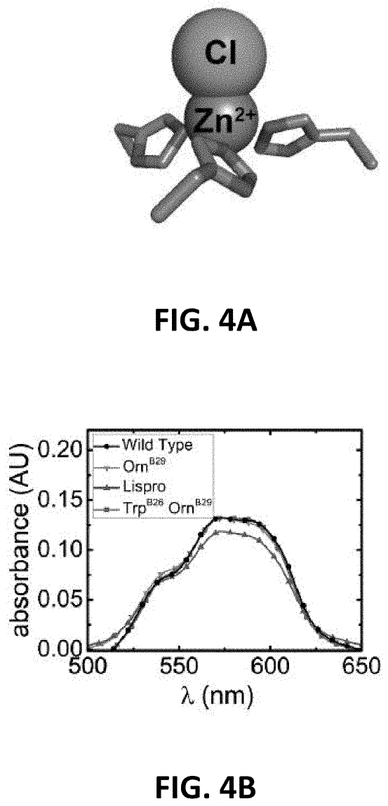

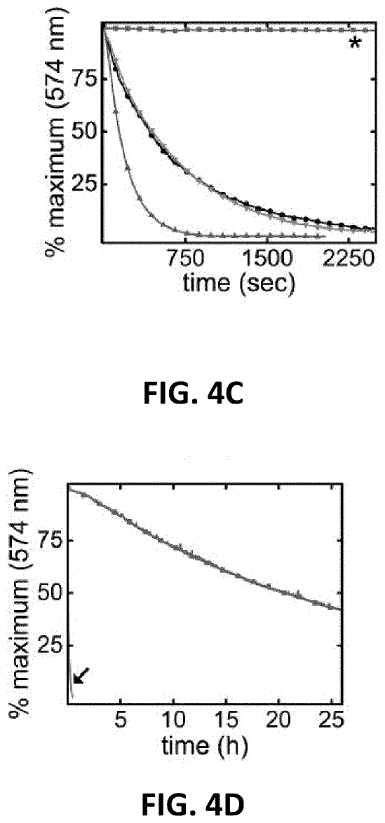

The present invention is directed toward a two-chain insulin analogue that provides protracted duration of action based on an aromatic B26 substitution. Although such a substitution may employ a natural or unnatural amino acid, our studies focused on substitution of Tyr^{B26} by Trp, a natural amino acid containing a bicyclic indole ring. The analogue was prepared by trypsin-mediated semi-synthesis using a synthetic octapeptide (sequence GFFWPOT, where “O” indicates the basic amino acid Omithine, introduced in place of Lysine to eliminate a tryptic site) together with the insulin fragment des-octapeptide[B23-B30]-insulin. The lifetime of the R₆ cobalt insulin hexamer is dramatically prolonged relative to R₆ cobalt hexamers formed by either wild-type human insulin or the immediate parent analogue Orn^{B29}-insulin (FIG. 4). This is the type of hexamer most commonly utilized in pharmaceutical formulations.

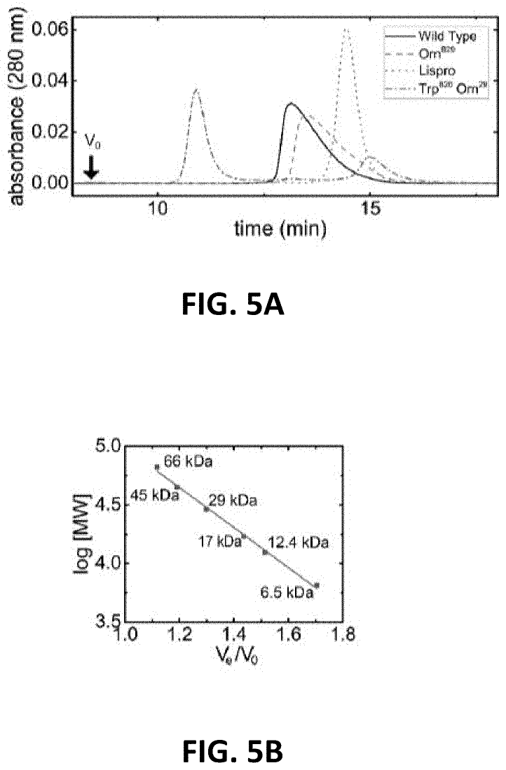

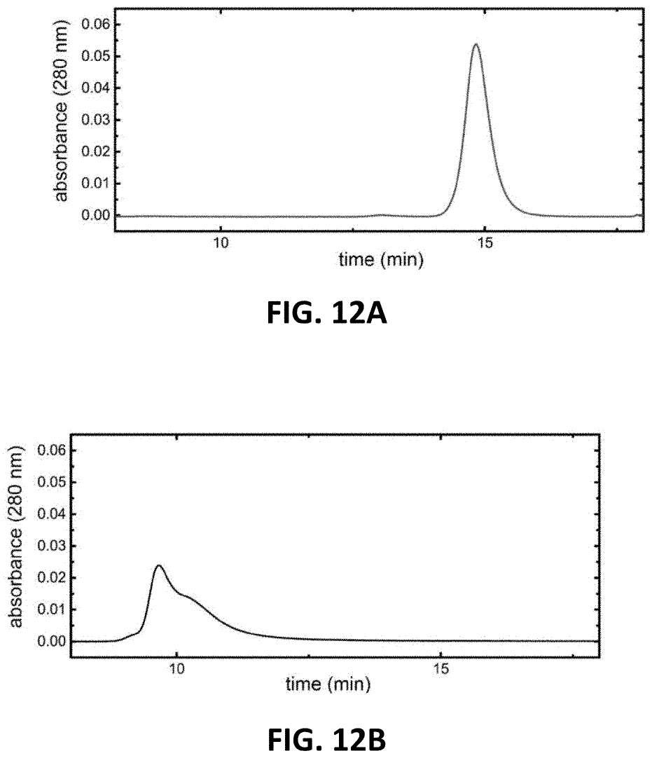

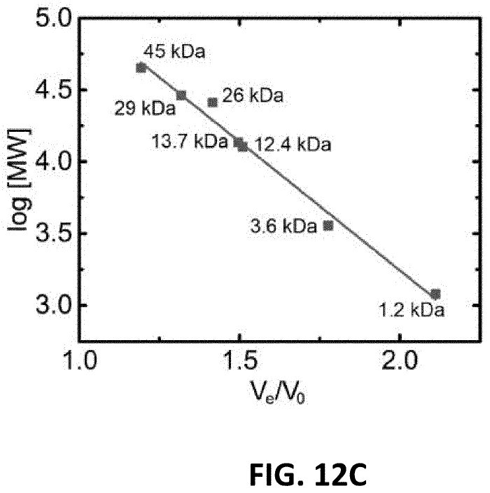

The affinity of this analog for the lectin-purified insulin receptor was ca. 50% relative to human insulin. Its potency in male Sprague-Dawley rats, rendered diabetic by streptozotocin, was greater than wild-type human insulin as shown below. The lifetime of the R₆ cobalt insulin hexamer (as an isomorphic model of the R₆ zinc insulin hexamer) was prolonged by at least 150-fold relative to wild-type human insulin or Orn^{B29}-insulin (column 2 in Table 1). Extent of zinc-dependent self-assembly, as probed by solvent-exclusion chromatography (SEC; FIG. 5), was also increased, suggesting thermodynamic stabilization relative to wild-type human insulin or Orn^{B29}-insulin (column 3 in Table 1; see also control studies in FIG. 12).

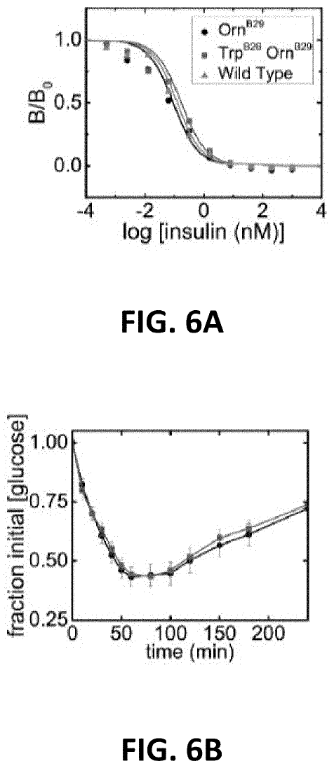

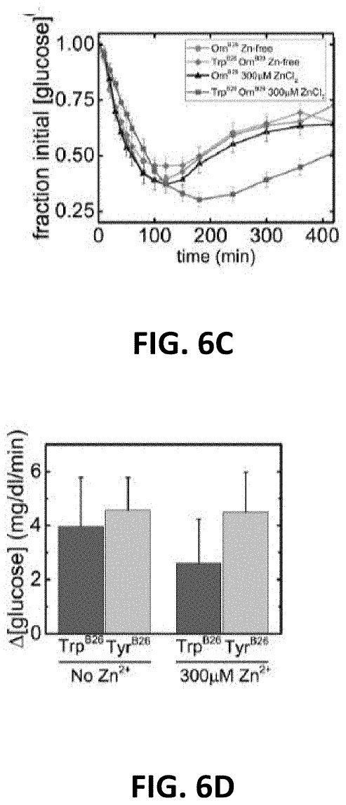

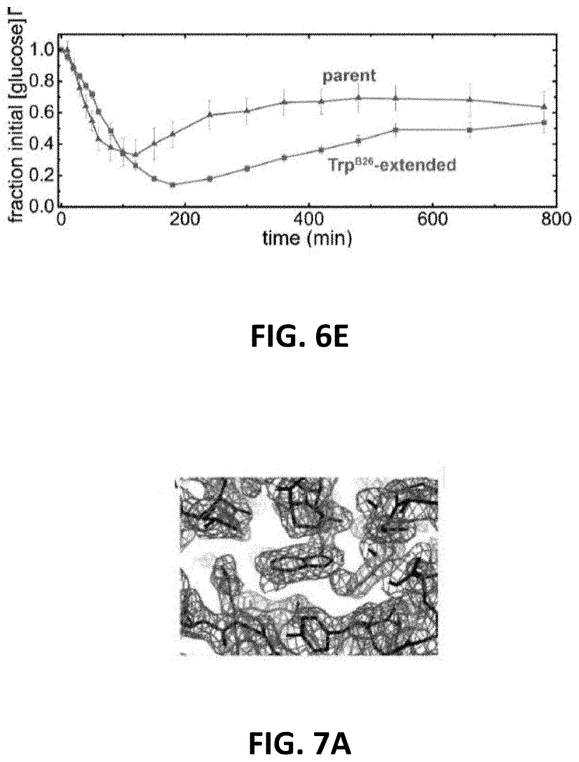

As is known in the art, hexamer assembly delays absorption of wild-type insulin from its subcutaneous injection site. To assess the onset and duration of Trp^{B26}, Orn^{B29}-insulin relative to Orn^{B29}-insulin, the pharmacodynamics (PD) profile of these proteins (made 0.15 mg/ml, corresponding to a monomer concentration of 27 μM and a putative hexamer concentration of 4.5 μM) were evaluated as zinc-free solutions or as pre-assembled phenol-stabilized R₆ hexamers in the presence of excess zinc ions (0.30 mM ZnCl₂; 70 zinc ions per hexamer). A zinc-dependent delay in onset of activity was observed on subcutaneous injection of Trp^{B26}, Orn^{B29}-insulin but not on injection of Orn^{B29}-insulin or wild-type human insulin (FIG. 6C). Whereas the latter PD profiles exhibited a nadir at ca. 120 min irrespective of zinc-ion concentration, the PD profile of Trp^{B26}, Orn^{B29}-insulin occurred at (i) 120 min in the absence of zinc ions and (ii) 150 min in the presence of 0.30 mM zinc ions with corresponding delays in rate of fall over the first 30 min (FIG. 6D). Together with the above in vitro results, these findings suggest that the prolonged lifetime of the Trp^{B26} R₆ hexamer (as inferred from the above kinetic studies of the Co²⁺-substituted hexamer) are responsible for the inferred zinc-dependent delay in SQ absorption.

As is also known in the art, insulin analogs with isoelectric points (pI) shifted to neutral pH generally exhibit prolonged activity due to precipitation in the SQ depot. To determine whether Trp^{B26} might further prolong the activity of such analogs, this substitution was introduced into a Gly^{A21}, Orn^{B29}, Orn^{B31}, Orn^{B32}-insulin. This “glargine-like” framework was designed to recapitulate the pI shift of glargine with greater ease of semisynthesis. The proteins (formulated at 0.6 mM with 0.3 mM ZnCl₂, corresponding to 3 zinc ions per hexamer) were each injected SQ in diabetic rats. The pI-shifted parent analog displayed peak activity at ca. 120 min with blood-glucose levels returning to baseline after about 360 min. By contrast, its Trp^{B26} derivative displayed a prolonged PD profile: peak activity occurred 180 min with slow return to baseline >800 min (FIG. 6E). Such a marked delay in peak activity was not observed in the parent glargine-like analog or the Trp^{B26} derivative when administered in the absence of zinc (not shown). These results imply that Trp^{B26} may favorably be incorporated into current basal analogs as a complementary mechanism of prolonged subcutaneous absorption.

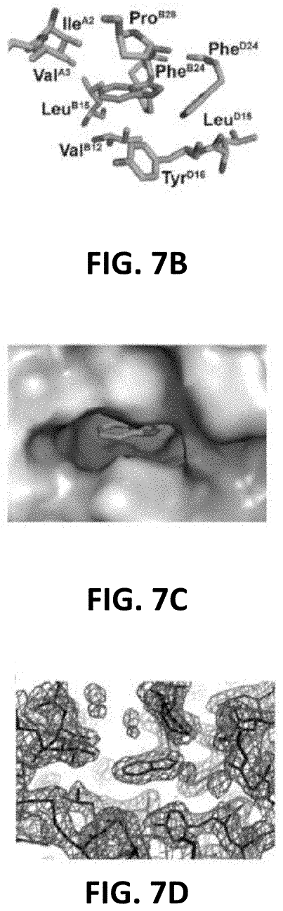

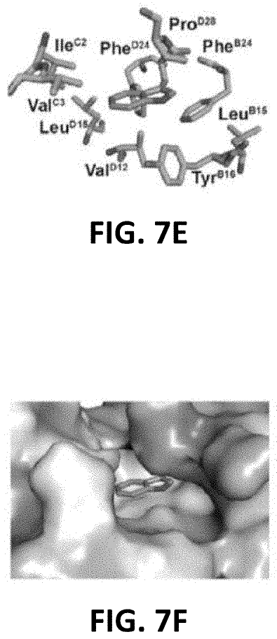

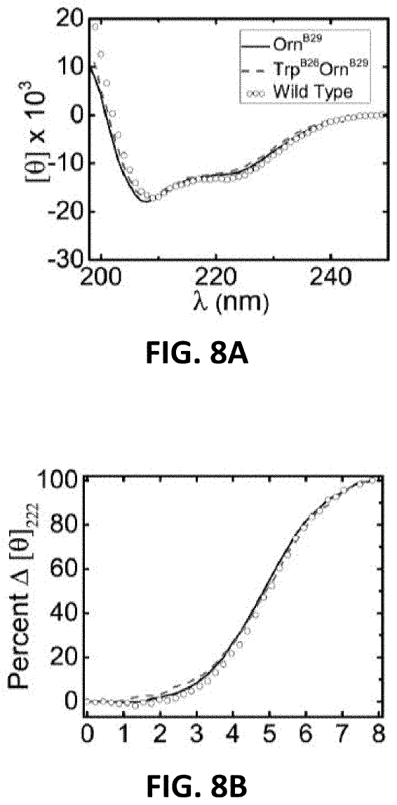

The crystal structure of Trp^{B26}, Orn^{B29}-insulin, determined as a T₃R^{f}₃ zinc hexamer, was essentially identical to that of human insulin in the same hexameric state (FIG. 7). The six-membered portion of the indole ring packs near Ile^{A2} whereas the indole NH group points toward the dimer interface. Substitution of Tyr^{B26} by Trp did not alter the thermodynamic stability of the insulin monomer as probed by CD-monitored denaturation at successive concentrations of guanidine hydrochloride (Table 2 and FIG. 8).

Although not wishing to be restricted by theory, molecular-mechanics calculations (using the standard CHARMM force field) suggested that substitution of Tyr^{B26} by Trp results in improved aromatic-aromatic interactions based on analysis of the variant crystal structure. The contribution of aromatic-aromatic interactions involving Trp^{B26} to the stability of the variant dimer interface of the T₃R^{f} hexamer was evaluated through calculation of non-bonded interaction energies among aromatic residues B16, B24, B25, and B26 in the TR^{f} dimer. In particular, based on aromatic-aromatic interactions alone, the Trp^{B26}, Orn^{B29} dimer displayed an increase in interaction energy of 1.4 kcal/mol relative to WT TR^{f} reference structure 1TRZ. Although the standard CHARMM empirical energy function, when applied to analyze the crystal structure of Trp^{B26} insulin, suggested that the electrostatic properties of the Trp side chain were the primary contributors to the increased stability of the dimer, this physical interpretation may reflect the limitations of the partial-charge representation. Indeed, preliminary ab initio QM simulations of a minimal model (consisting of two aromatic rings in vacuo) predict that enhanced Van der Waals interactions may also make a significant contribution.

TABLE 1
Self-association properties of insulin analogs.

| | t_{1/2} hexamer dissociation | calculated MW by SEC^{a} |
|---|---|---|
| analog | (min ± SD ) | (kDa) |
| Wild Type | 7.7 (±1.3) | 9.7 |
| Lispro^{b} | 4.6 (±0.3) | 5.1 |
| Orn^{B29 c} | 8.2 (±0.8) | 8.2 |
| Trp^{B26}, Orn^{B29} | 1.2(±0.3) × 10³ | 28.0, 4.0 |

^{a}Proteins were made 0.6 mM in a buffer containing ZnCl₂ at a ratio of 2 zinc ions per insulin hexamer and applied to SEC column as described in Methods. Masses were calculated from the plot in FIG. 4B.
^{b}“Lispro” describes insulin analogues containing Pro^{B28}→Lys and Lys^{B29}→Pro substitutions. These substitutions impair dimerization (28, 29).
^{c} Use of Orn simplified trypsin-catalyzed semisynthesis (33).

TABLE 2
Thermodynamic stabilities of insulin analogs.

| | ΔGᵤ^{a} | C_{mid} | m^{b} |
|---|---|---|---|
| Analog | (kcal mol⁻¹) | (M) | (kcal mol⁻¹ M⁻¹) |
| Wild-type | 3.4 ± 0.1^{c} | 5.0 ± 0.1 | 0.68 ± 0.02 |
| Orn^{B29} | 3.3 ± 0.1 | 4.9 ± 0.1 | 0.67 ± 0.01 |
| Trp^{B26}, Orn^{B29} | 3.3 ± 0.1 | 5.1 ± 0.2 | 0.64 ± 0.03 |

^{a}Parameters were inferred from CD-detected guanidine denaturation data by application of a two-state model; uncertainties represent fitting errors for a given data set.
^{b}The m-value (slope Δ(G)/Δ(M)) correlates with surface area exposed on denaturation.
^{c}Analysis of replicates of Trp^{B26}, Orn^{B29}-insulin, parent Orn^{B29}-insulin, and WT samples indicated that experimental standard errors were equal to or less than the above fitting errors: ±0.1 kcal mol−1 (ΔGᵤ), ±0.1M (C_{mid}), and ±0.01 kcal mol−1 M−1 (m).

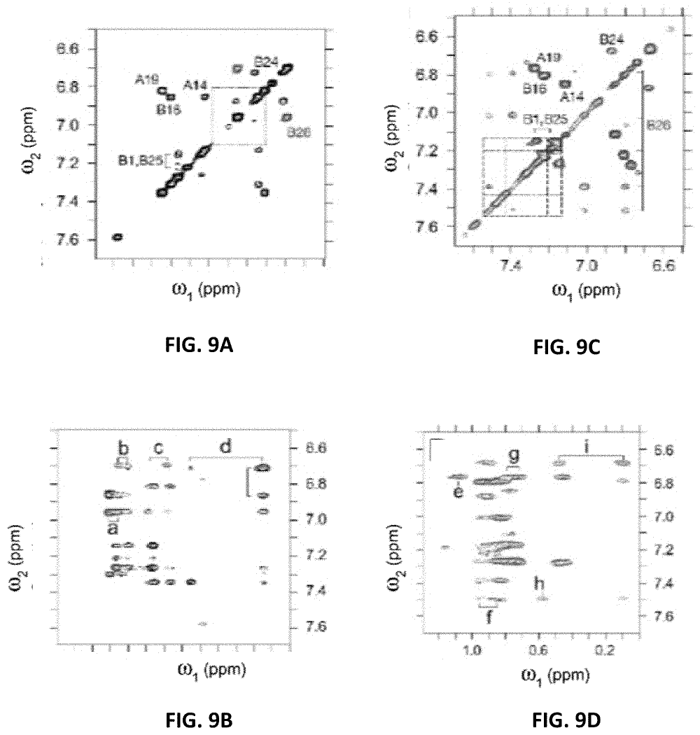

Spectroscopic probes revealed native-like structure and thermodynamic stability of Trp^{B26} analogues in solution. The native-like crystal structure of Trp^{B26}, Orn^{B29}-insulin is in accordance with its unperturbed circular dichroism (CD) spectrum and thermodynamic stability under monomeric conditions (FIG. 8A). Free energies of unfolding (ΔGᵤ 3.3±0.1) kcal/mole at 25° C. as inferred from two-state modeling of chemical denaturation (33)) were indistinguishable due to small and compensating changes in transition midpoint and slope (m value) (FIG. 8B, Table 2). Further evidence that the crystal structure extends to the monomer in solution was provided by 2D ¹H-NMR studies of Trp^{B26} substituted within an engineered insulin monomer (insulin lispro). Whereas the spectrum of insulin lispro (at pD 7.6 and 37° C.) exhibits sharp resonances for each aromatic spin system (FIG. 9A), as expected for a monomeric analog, the spectrum of its Trp^{B26} derivative exhibits broadening of resonances at the dimer interface (B16, B24-B26). The latter spin systems can be observed on TOCSY spectrum (FIG. 9C) but not in the corresponding DQF-COSY spectrum due to antiphase cancellation. Like the aromatic ring Tyr^{B26} in spectra of insulin lispro (FIGS. 9A, 9B), the indole ring exhibited regiospecific nonlocal nuclear Overhauser enhancements (NOEs) from its six-member moiety to the methyl resonances of Val^{B12} and Ile^{A2} (FIGS. 9B-9D).

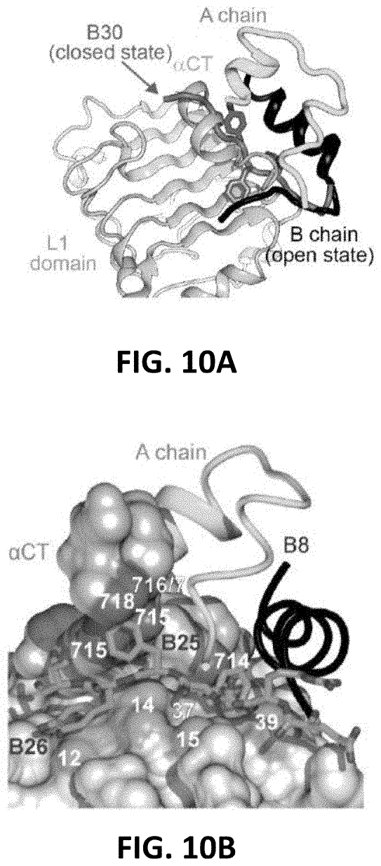

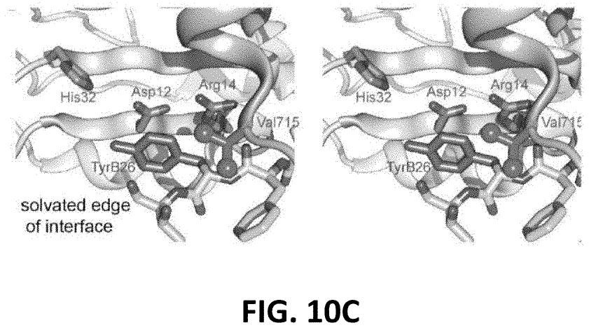

The pattern of secondary shifts in the variant is similar to that in the parent monomer. In particular, the aromatic ¹H-NMR resonances of Trp^{B26} (red cross peaks in FIG. 9C) exhibit upfield features (relative to Trp in the isolated B23-B30 octapeptide; dashed lines) similar to those of Tyr^{B26} in the parent spectrum (purple cross peaks in FIG. 9A versus dotted lines). Dilution of the Trp^{B26} sample partially mitigated resonance broadening but preserved these trends in dispersion. Indole-specific NOEs indicated that the side chain assumes one predominant and asymmetric conformation within a native-like crevice between A- and B-chain α-helices. Because Tyr^{B26} undergoes rapid ring rotation about the C_{β}-C_{γ} bond axis (“ring flips”), analogous side-chain specific NOEs (inferred in prior studies from molecular modeling) cannot be observed directly. Modeling based on the co-crystal structure of wild-type insulin bound to the “micro-receptor” fragment of the receptor ectodomain (FIG. 10) suggests that Trp^{B26} will contact the receptor surface essentially as described for Tyr^{B26}.

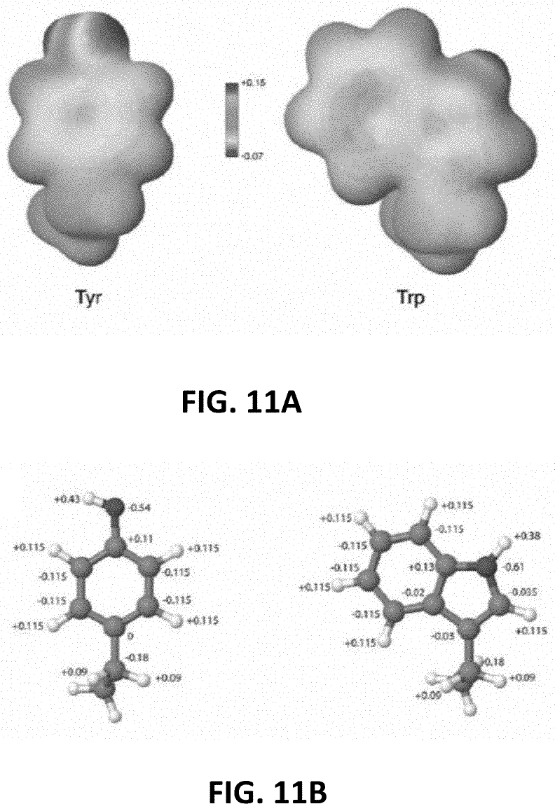

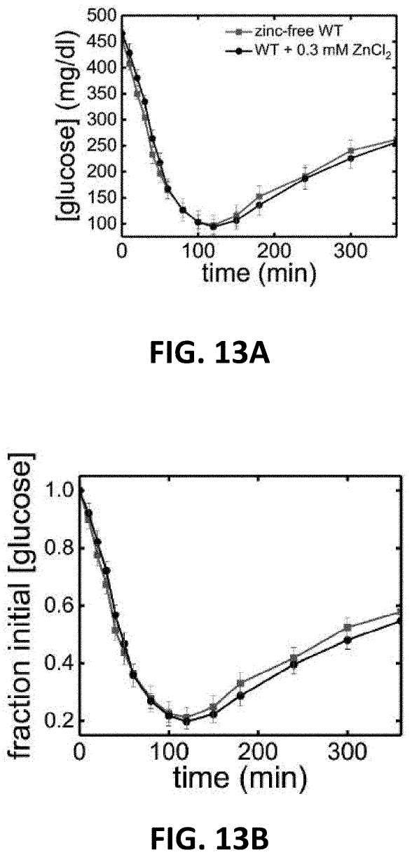

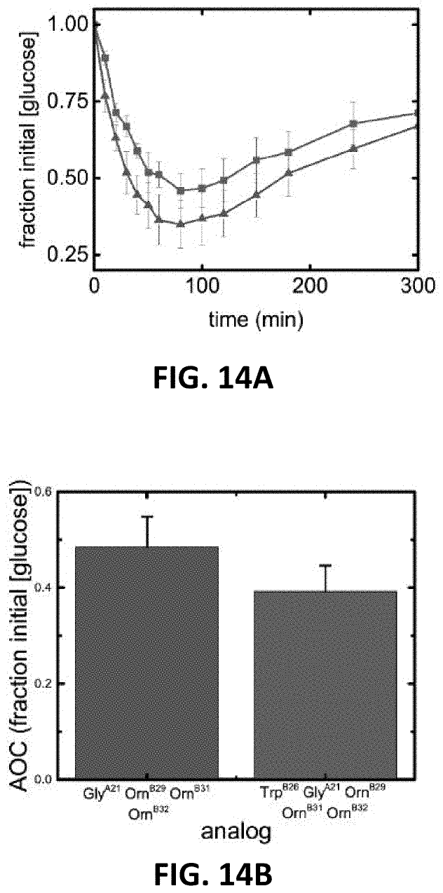

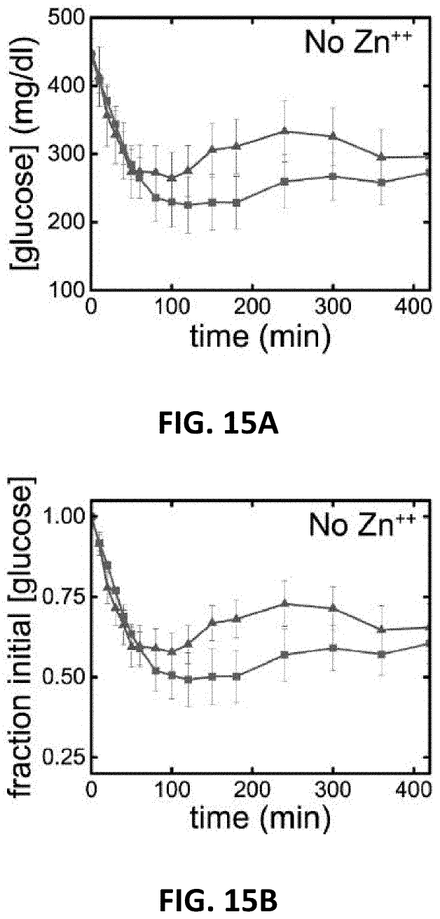

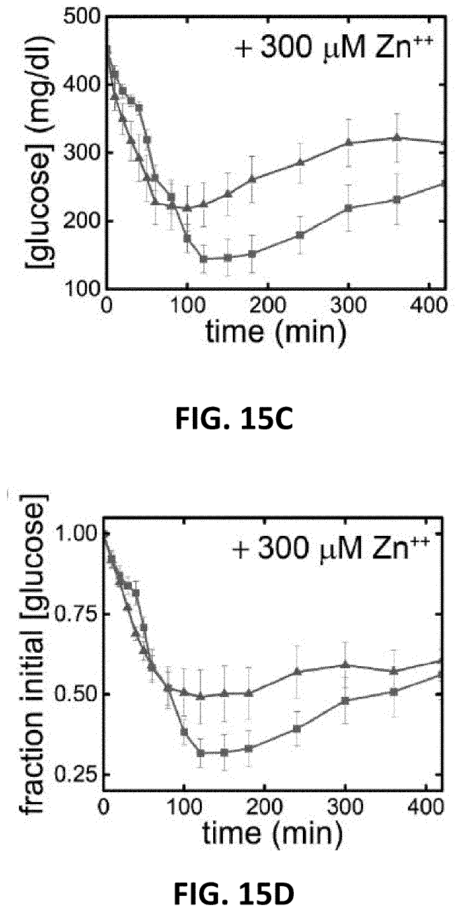

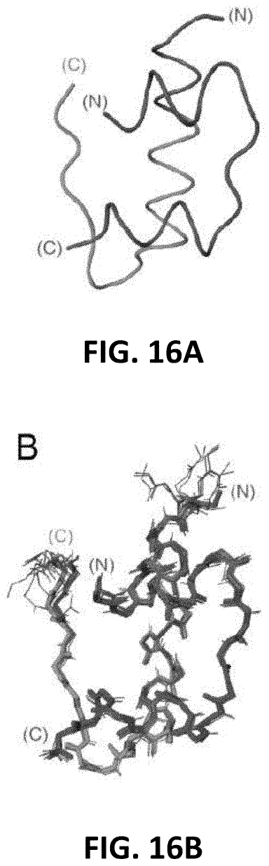

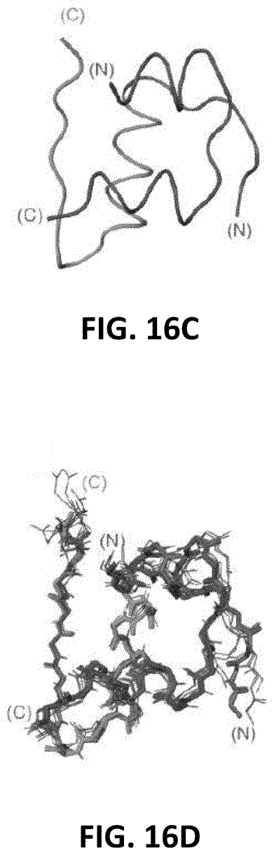

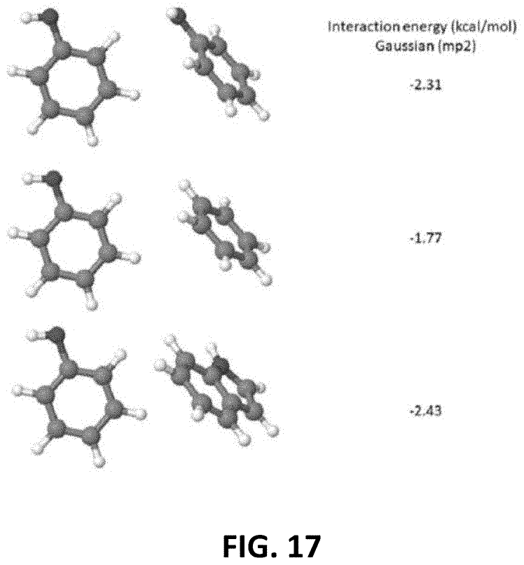

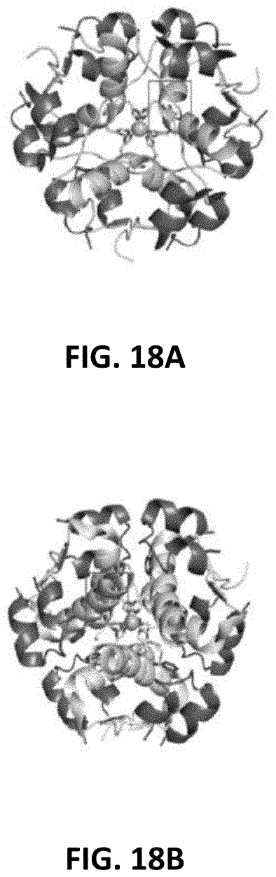

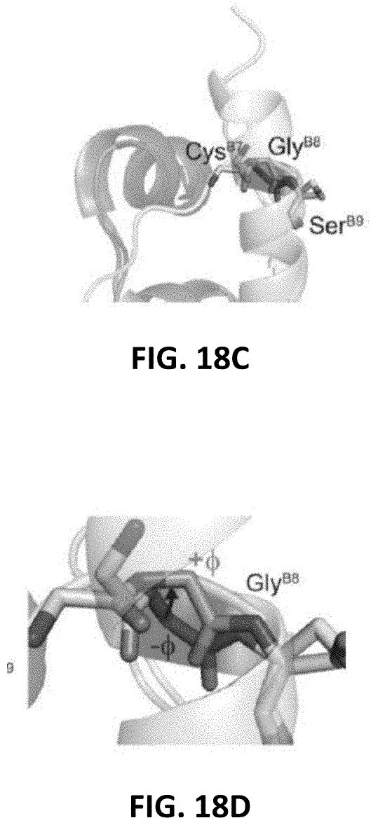

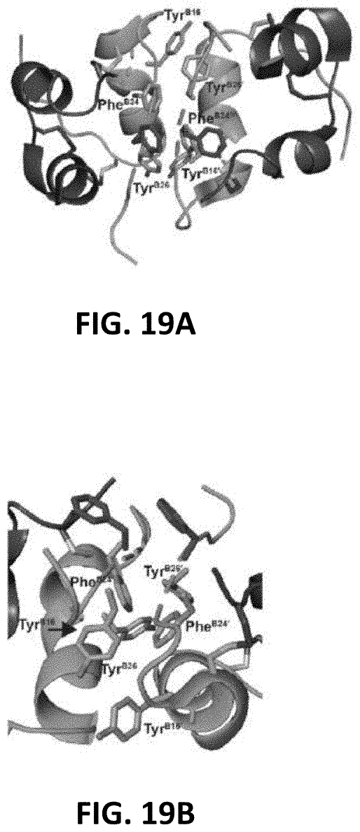

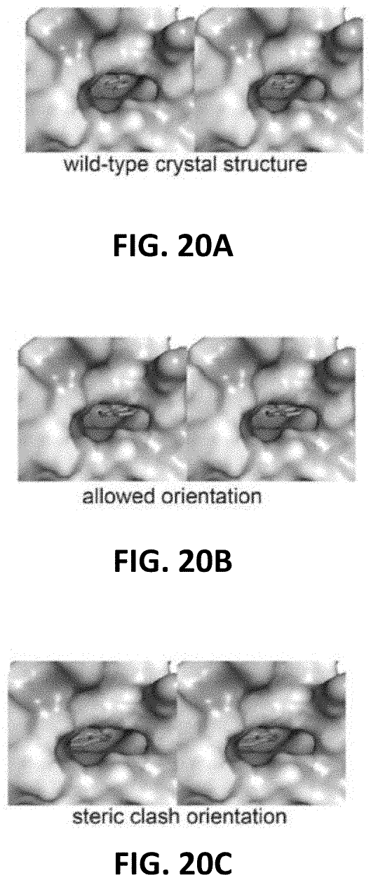

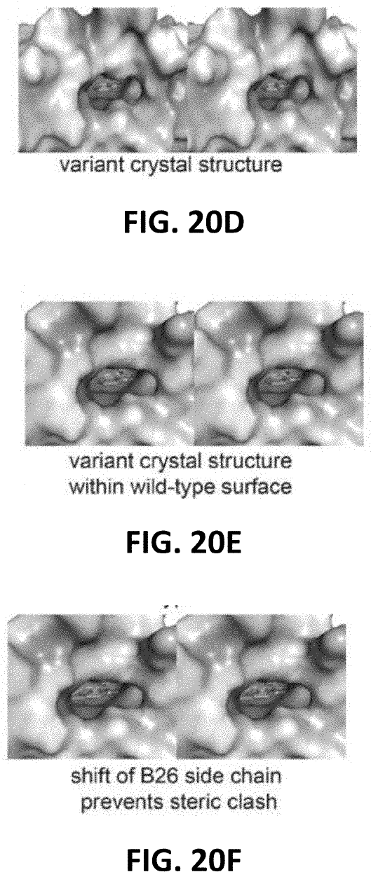

Control data are provided in FIGS. 11-13. FIG. 11 provides the standard partial charges for Tyrosine and Tryptophan employed in the CHARMM empirical energy function. Although our invention and its usefulness are not dependent on theory, the partial-charge representation of aromatic rings in CHARMM suggests that Trp^{B26} enhances aromatic-aromatic interactions at the dimer interface relative to Tyr^{B26}. The SEC data shown in FIG. 12 enabled calibration of the column. Control rat studies in FIG. 13 demonstrate that wild-type human insulin exhibits similar PD profiles in the presence or absence of zinc ions.

We envisage that a diversity of non-standard aromatic side chains may function as well as, or better than, Trp when introduced at position B26, to stabilize the insulin dimer and to prolong the lifetime of the zinc insulin hexamer. Trypsin-mediated semi-synthesis in principle enables the convenient and cost-effective incorporation of such residues via a synthetic octapeptide. Modern computational chemistry promises to enable a virtual screen of an in silico library of such aromatic systems.

It is also envisioned that Trp^{B26}-containing analogues may also be made with A- and B-domain sequences derived from animal insulins, such as porcine, bovine, equine, and canine insulins, by way of non-limiting examples. In addition or in the alternative, the insulin analogue of the present invention may contain modifications described above as known in the art to confer protracted action: i.e., Arg^{B31}-Arg^{B32} or other amino-acid additions or substitutions introduced to shift the iso-electric point of the resulting insulin analogue to near-neutrality and hence permit iso-electric precipitation on subcutaneous injection; (ii) acylation of the epsilon-amino-group of Lysine at position B29 or its modification by a 16-carbon fatty di-carboxylic acid attached via a glutamic acid spacer; and/or (iii) covalent addition of poly-ethylene-glycol to the insulin analogue. It is also encompassed within the scope of the present invention that the Trp^{B26} or suitable unnatural aromatic amino-acid residues at position B26 may be placed within a single-chain insulin analogue containing a foreshortened C-domain of 4-12 residues to likewise promote their self-assembly.

Furthermore, in view of the similarity between human and animal insulins and in view of the use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Diaminobutyric acid, or Diaminopropionic acid). Lys may also be replaced by the neutral aliphatic isostere Norleucine (Nle), which may in turn be substituted by analogues containing shorter aliphatic side chains (Aminobutyric acid or Aminopropionic acid).

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A-chain of human insulin is provided as SEQ ID NO: 2.

(human A-chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B-chain of human insulin is provided as SEQ ID NO: 3.

(human B-chain)
SEQ ID NO: 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of analogue of the human B-chain containing Trp^{B26} is shown as SEQ. ID NO: 4.

(modified human B-chain)
SEQ ID NO: 4
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Trp-Thr-Pro-Lys-Thr

The amino-acid sequence of analogue of the human B-chain containing Trp^{B26} in the context of a di-Arg-extended B chain is shown as SEQ. ID NO: 5.

(modified human B-chain)
SEQ ID NO: 5
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Trp-Thr-Pro-Lys-Thr-Arg-Arg

The amino-acid sequence of analogue of the human B-chain containing Trp^{B26} in the context of a Lys-modified B chain is shown as SEQ. ID NO: 6.

(modified human B-chain)
SEQ ID NO: 6
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Trp-Thr-Pro-Lys*

Where Lys* indicates an ε-N-acylated Lysine or its modification by a 16-carbon fatty di-carboxylic acid attached via a glutamic acid spacer and where Thr^{B30} may optionally be absent.

The amino-acid sequence of analogue of the human B-chain containing Trp^{B26} in the context of Gln^{B13} is shown as SEQ. ID NO: 7.

(modified human B-chain)
SEQ ID NO: 7
Phe-Val-Asn-Gln-His-Leu-Cys-Gly-Ser-His-Leu-Val-
Gln-Ala-Leu-Tyr-Leu-Val-Cys-Gly-Glu-Arg-Gly-Phe-
Phe-Trp-Thr-Pro-Lys-Thr

The amino-acid sequence of a variant A-chain of human insulin containing Gln^{A8} is provided as SEQ ID NO: 8.

(variant human A-chain)
SEQ ID NO: 8
Gly-Ile-Val-Glu-Gln-Cys-Cys-Gln-Ser-Ile-Cys-Ser-
Leu-Tyr-Gln-Leu-Glu-Asn-Tyr-Cys-Asn

The amino-acid sequence of a variant A-chain of human insulin containing His^{A8} is provided as SEQ ID NO: 9.

(variant human A-chain)
SEQ ID NO: 9
Gly-Ile-Val-Glu-Gln-Cys-Cys-Xaa₁-Ser-Ile-Cys-Ser-
Leu-Tyr-Gln-Leu-Glu-Asn-Tyr-Cys-Xaa₂

Where Xaa₁ indicates Arg, His or Gln; and where Xaa₂ indicates Ala, Asn, or Gly.

The amino-acid sequence of a variant A-chain of human insulin containing paired substitutions His^{A8} and His^{A8} is provided as SEQ ID NO: 10.

(variant human A-chain)
SEQ ID NO: 10
Gly-Ile-Val-His-Gln-Cys-Cys-His-Ser-Ile-Cys-Ser-
Leu-Tyr-Gln-Leu-Glu-Asn-Tyr-Cys-Xaa₁

Where Xaa₁ indicates Ala, Asn, or Gly.

The amino-acid sequence of a variant A-chain of human insulin containing paired substitutions His^{A8} and His^{A8} with the addition of Gly^{A21} is provided as SEQ ID NO: 11.

(variant human A-chain)
SEQ ID NO: 11
Gly-Ile-Val-His-Gln-Cys-Cys-His-Ser-Ile-Cys-Ser-
Leu-Tyr-Gln-Leu-Glu-Asn-Tyr-Cys-Xaa₁

Where Xaa₁ indicates Ala, Asn, or Gly.

The amino acid sequence of a variant B-chain of human insulin containing an Orn^{B29} substitution is provided as SEQ ID NO: 12.

(Orn^{B29})
SEQ ID NO: 12
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Xaa-Thr

Where Xaa is Omithine (Orn).

The amino acid sequence of a variant B-chain of human insulin containing a Trp^{B26} substitution and an Orn^{B29} substitution is provided as SEQ ID NO: 13.

(Trp^{B26}, Orn^{B29})
SEQ ID NO: 13
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Trp-Thr-Pro-Xaa-Thr

Where Xaa is Omithine (Orn).

The amino acid sequence of the B-chain of lispro insulin, containing a Lys^{B28} substitution and a Pro^{B29} substitution, is provided as SEQ ID NO: 14.

(lispro B-chain)
SEQ ID NO: 14
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Lys-Pro-Thr

The amino acid sequence of a variant B-chain of human insulin containing an Orn^{B29} substitution and a C-terminal extension of Orn-Orn is provided as SEQ ID NO: 15.

(Orn^{B29}, Orn^{B31}, Orn^{B32})
SEQ ID NO: 15
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Xaa-Thr-Xaa-Xaa

Where Xaa is Omithine (Orn).

The amino acid sequence of a variant B-chain of human insulin containing a Trp^{B26} substitution, an Orn^{B29} substitution and a C-terminal extension of Orn-Orn is provided as SEQ ID NO: 16.

(Trp^{B26}, Orn^{B29}, Orn^{B31}, Orn^{B32})
SEQ ID NO: 16
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Trp-Thr-Pro-Xaa-Thr-Xaa-Xaa

Where Xaa is Omithine (Orn).

The amino-acid sequence of a variant A-chain of human insulin containing a Gly^{A21} substitution is provided as SEQ ID NO: 17.

(variant human A-chain)
SEQ ID NO: 17
Gly-Ile-Val-Glu-Gln-Cys-Cys-Thr-Ser-Ile-Cys-Ser-
Leu-Tyr-Gln-Leu-Glu-Asn-Tyr-Cys-Gly.

What is claimed is:

1. An insulin analogue comprising an insulin B-chain polypeptide containing a Trp substitution at position B26 relative to the sequence of wild-type insulin.

2. The insulin analogue of claim 1, wherein the analogue has an iso-electric point between 6.5 and 8.0.

3. The insulin analogue of claim 1, wherein the B-chain polypeptide additionally comprises an Orn substitution at position B29 relative to wild-type insulin.

4. The insulin analogue of claim 3, wherein the B-chain polypeptide additionally comprises a C-terminal extension of one or two basic amino acids.

5. The insulin analogue of claim 4, wherein the C-terminal extension of the B-chain polypeptide consists of Arg residues at positions B31 and B32 relative to wild-type insulin.

6. The insulin analogue of claim 4, additionally comprising an insulin A-chain polypeptide containing a Gly substitution at position A21 relative to wild-type insulin.

7. The insulin analogue of claim 1, wherein the B-chain polypeptide additionally comprises a Gln substitution at position B13 relative to wild-type insulin.

8. The insulin analogue of claim 1, additionally comprising an insulin A-chain polypeptide containing a His or Arg substitution at position A8 relative to wild-type insulin.

9. The insulin analogue of claim 8, wherein the insulin A-chain polypeptide additionally comprises a Gly or Ala substitution at position A21 relative to wild-type insulin.

10. The insulin analogue of claim 1, formulated in the presence of zinc ions at a molar ratio of 2.2-10 zinc ions per six insulin analogue monomers.

11. The insulin analogue of claim 10, formulated in the presence of zinc ions at a molar ratio of 2.0-3.0 zinc ions per six insulin analogue monomers.

12. The insulin analogue of claim 1, wherein the B-chain polypeptide additionally comprises a C-terminal extension of one or two basic amino acids.

13. The insulin analogue of claim 12, wherein the C-terminal extension of the B-chain polypeptide consists of Arg residues at positions B31 and B32 relative to wild-type insulin.

14. The insulin analogue of claim 13, additionally comprising an insulin A-chain polypeptide containing a Gly substitution at position A21 relative to wild-type insulin.

15. The insulin analogue of claim 14, wherein the B-chain polypeptide additionally comprises a Gln substitution at position B13 relative to wild-type insulin

16. A method of lowering the blood sugar level of a patient in need thereof, the method comprising administering a physiologically effective amount of insulin analogue or a physiologically acceptable salt thereof to a patient, wherein the insulin analogue comprises an insulin B-chain polypeptide containing a Trp substitution at position B26 relative to the sequence of wild-type insulin.

17. The method of claim 16, wherein the B-chain polypeptide additionally comprises an Orn substitution at position B29 relative to wild-type insulin.

18. The method of claim 17, wherein the B-chain polypeptide additionally comprises a C-terminal extension of the B-chain polypeptide consisting of Arg residues at positions B31 and B32 relative to wild-type insulin.

19. The method of claim 18, wherein the insulin analogue additionally comprises an insulin A-chain polypeptide containing a Gly substitution at position A21 relative to wild-type insulin.

20. The method of claim 16, wherein the B-chain polypeptide additionally comprises a C-terminal extension of the B-chain polypeptide consisting of Arg residues at positions B31 and B32 relative to wild-type insulin.


SEQUENCE LISTING (17 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 30 aa, Artificial Sequence
  SYNTHETIC CONSTRUCT
FVNQHLCGSHLVEALYLVCGERGFFWTPKT

SEQ ID NO: 5 - protein, 32 aa, Artificial Sequence
  SYNTHETIC CONSTRUCT
FVNQHLCGSHLVEALYLVCGERGFFWTPKTRR

SEQ ID NO: 6 - protein, 30 aa, Artificial Sequence
  SYNTHETIC CONSTRUCT
  MISC_FEATURE (29)..(29): xaa is an epsilon-N-acylated Lysine or its modification by a 16-carbon fatty dicarboxylic acid attached via a glutamic acid spacer
  MISC_FEATURE (30)..(30): xaa is Thr or is absent
FVNQHLCGSHLVEALYLVCGERGFFWTPXX

SEQ ID NO: 7 - protein, 30 aa, Artificial Sequence
  SYNTHETIC CONSTRUCT
FVNQHLCGSHLVQALYLVCGERGFFWTPKT

SEQ ID NO: 8 - protein, 21 aa, Artificial Sequence
  SYNTHETIC CONSTRUCT
GIVEQCCQSICSLYQLENYCN

SEQ ID NO: 9 - protein, 21 aa, Artificial Sequence
  SYNTHETIC CONSTRUCT
  FEATURESMISC (8)..(8): xaa is Arg, His or Gln
  MISC_FEATURE (21)..(21): xaa is Ala, Asn, or Gly
GIVEQCCXSICSLYQLENYCX

SEQ ID NO: 10 - protein, 21 aa, Artificial Sequence
  SYNTHETIC CONSTRUCT
  MISC_FEATURE (21)..(21): xaa is Ala, Asn, or Gly
GIVHQCCHSICSLYQLENYCX

SEQ ID NO: 11 - protein, 21 aa, Artificial Sequence
  ARTIFICIAL CONSTRUCT
  MISC_FEATURE (21)..(21): xaa is Ala, Asn, or Gly
GIVHQCCHSICSLYQLENYCX

SEQ ID NO: 12 - protein, 30 aa, Artificial Sequence
  SYNTHETIC CONSTRUCT
  MISC_FEATURE (29)..(29): xaa is Orn
FVNQHLCGSHLVEALYLVCGERGFFYTPXT

SEQ ID NO: 13 - protein, 30 aa, Artificial Sequence
  SYNTHETIC CONSTRUCT
  MISC_FEATURE (29)..(29): xaa is Orn
FVNQHLCGSHLVEALYLVCGERGFFWTPXT

SEQ ID NO: 14 - protein, 30 aa, Artificial Sequence
  SYNTHETIC CONSTRUCT
FVNQHLCGSHLVEALYLVCGERGFFYTKPT

SEQ ID NO: 15 - protein, 32 aa, Artificial Sequence
  SYNTHETIC CONSTRUCT
  MISC_FEATURE (29)..(29): xaa is Orn
  MISC_FEATURE (31)..(31): xaa is Orn
  MISC_FEATURE (32)..(32): xaa is Orn
FVNQHLCGSHLVEALYLVCGERGFFYTPXTXX

SEQ ID NO: 16 - protein, 32 aa, Artificial Sequence
  SYNTHETIC CONSTRUCT
  MISC_FEATURE (29)..(29): xaa is Orn
  MISC_FEATURE (31)..(31): xaa is Orn
  MISC_FEATURE (32)..(32): xaa is Orn
FVNQHLCGSHLVEALYLVCGERGFFWTPXTXX

SEQ ID NO: 17 - protein, 21 aa, Artificial Sequence
  SYNTHETIC CONSTRUCT
GIVEQCCTSICSLYQLENYCG